(19) United States
(12) Patent Application Publication
(10) Pub. No.: US 2021/0022645 A1
(43) Pub. Date: Jan. 28, 2021

(54) INDICATOR DEVICE, MEASURING APPARATUS WITH INDICATOR DEVICE AND COMPUTER PROGRAM PRODUCT FOR EVALUATING THE INDICATOR DEVICE

(71) Applicant: mHealth Technologies GmbH, Hamburg (DE)

(72) Inventors: Maxim Andreev, Hamburg (DE); Ferdinand Yussupov, Hamburg (DE)

(21) Appl. No.: 16/982,296

(22) PCT Filed: Mar. 21, 2019
(86) PCT No.: PCT/EP2019/057166

§ 371 Date: Sep. 18, 2020

(30) Foreign Application Priority Data
Mar. 21, 2018 (DE) 10 2018 002 318.5

(51) Int. Cl. A61B 5/087; A61B 5/09; A61B 5/00

(52) U.S. Cl. CPC A61B 5/0871; A61B 5/4842; A61B 5/09

(57) ABSTRACT

An indicator device represents a measured value and includes a first component and a second component. The first component includes a machine-readable first reference marker, a machine-readable second reference marker spaced from the first reference marker and a guide for the second component. The first reference marker defines a first reference point and the second reference marker defines a second reference point. The position and orientation of a predetermined path are defined by the first reference point and the second reference point. The second component can be positioned along the guide by a displacement force. The second component alone, or together with the first component, forms a machine-readable value indicator. The measured value is represented by a position of the value indicator along the predetermined path. The disclosure also relates to a measuring apparatus comprising the indicator device. The disclosure also relates to a computer program product for evaluating the indicator device of the measuring apparatus.

### CROSS-REFERENCE TO RELATED APPLICATIONS

This application is the US national phase of PCT/EP2019/057166 filed on Nov. 21, 2017, which claims priority over German Application No. 102018002318.5 which was filed on Mar. 21, 2018.

### TECHNICAL FIELD

The present disclosure relates to an indicator device for mechanically representing a measured value. The disclosure also relates to a measuring apparatus comprising the indicator device and a computer program product for evaluating the indicator device.

### BACKGROUND

Nowadays, mechanically measured values (e.g. forces, lengths, displacements, etc.) are often measured with corresponding electronic measuring elements (sensors, e.g. strain gauges, electromagnetic travel sensors, etc.) and digitized directly in the measuring apparatus. This unquestionably leads to relatively good accuracy of the measured values. The mechanical complexity of the measuring apparatus can also often be reduced. In addition, the subsequent processing of the measured values is significantly simplified. In this respect, a trend towards electrification and digitization of formerly purely mechanical measuring apparatus has been apparent for many years.

However, there are a few measurement tasks that are carried out in very price-sensitive fields. In these situations, even a very inexpensive electronic measuring apparatus can be prohibitively expensive for a consumer or a service provider.

One such example are peak flow meters. Peak flow meters measure the peak expiratory flow. The peak flow, also called peak expiratory flow (PEF), is a measurement value in medicine that records the maximum speed of expiration of a person. This value is measured in liters per minute (L/min). As with all measured respiratory flow rates, the measurement result strongly depends on the cooperation of the patient and the correct execution of the breathing maneuver.

Peak flow meters measure the air flow through the bronchi and are therefore a measure of pulmonary function. Peak flow meters allow physicians to track changes in the patient's breathing condition and diagnose potential or existing breathing difficulties. Patients also use peak flow meters outside of a physician's office to regularly monitor their own condition. Peak flow meters are also and especially used to monitor common respiratory diseases such as asthma and chronic obstructive pulmonary diseases (COPD) and to support athletes (competitive athletes such as cross-country skiers, competitive swimmers, etc.) and recipients of lung transplants in monitoring lung performance

Digital/electronic peak flow meters include a suitable sensor, a corresponding control and evaluation unit and usually also a display. These components make an electrical/digital peak flow meter comparatively complex to construct and expensive to purchase. Electronic/digital peak flow meters are often not waterproof. Thus, they are difficult or impossible to clean.

While mechanical peak flow meters of the known type are indeed very inexpensive, they are limited in minimum size by the minimum useful resolution of a scale assigned to the indicator. In other words, mechanical peak flow meters are comparatively large, unwieldy and difficult to carry because the indicator has to be large enough for a human to be able to read it easily. Another disadvantage is the high effort involved in long-term monitoring of the success of the therapy, including time-consuming manual documentation. Mechanical peak flow meters are normally not vulnerable to water, but they often have joints and/or cavities in which, for example, saliva and/or other dirt can accumulate during use. Hygienic cleaning of the hard-to-reach areas of the measuring apparatus is difficult then.

### BRIEF SUMMARY

The disclosed device eliminates or at least reduces disadvantages both of mechanical measuring apparatus for measuring a measured value and of electronic measuring apparatus for measuring a measured value.

Among other things, an indicator device for representing a measured value is provided. The (mechanically) measured value can—purely as an example—be the displacement of an air resistance element arranged in an air duct through which the air flows in an articulated manner.

The indicator device comprises a first component and a second component. The second component is configured to be movable with respect to the first component.

The first component comprises a machine-readable first reference marker, a machine-readable second reference marker spaced from the first reference marker, and a guide for the second component.

The first and the second components may each comprise more than one component parts.

The first reference marker directly or indirectly defines a first reference point. The second reference marker directly or indirectly defines a second reference point. A position and an orientation of a predetermined path are defined by the first reference point and the second reference point. For the predefined path, for example, corresponding construction rules based on the first and/or the second reference point may be known. In the course of the evaluation of the indicator device, the known construction rules may be used to determine the predetermined path.

The second component is positionable along the guide by a displacement force. The second component alone or together with the first component forms a machine-readable value indicator.

The measured value is represented by a position of the value indicator along the predetermined path. Accordingly, corresponding values are associated with the positions along the predetermined path.

The above aspects enable or facilitate the evaluation of the indicator device by a mobile terminal with an associated camera. Neither the indicator device itself nor a measuring apparatus with the indicator device are dependent on electrical and/or electronic components. The indicator device and the measuring apparatus may be purely mechanical.

According to an advantageous aspect, the first reference marker, the second reference marker and/or the value indicator may each have at least two marker surfaces. In particular, the reference markers may advantageously have four marker surfaces. The marker surfaces of the respective reference marker or value indicator may define an associated reference point.

According to a further advantageous aspect, at least two of the marker surfaces of the respective reference marker or value indicator may each adjoin at least one further of the marker surfaces. As a result, a marker surface boundary is formed between two adjacent marker surfaces. The reference point associated with the respective reference marker or value indicator may preferably be defined by a common intersection point of at least two marker surface boundaries.

According to a further advantageous aspect, a machine-readable third reference marker with a third reference point may be associated with the first reference marker. The first reference point, together with the third reference point and the second reference point, may define the position and the orientation of the predetermined path and may additionally be used to at least partially compensate for a distortion resulting from a tilt of the camera.

Advantageously, a machine-readable fourth reference marker with a fourth reference point may further be associated with the second reference marker. The position and the orientation of the predetermined path may be defined by the first reference point together with the third reference point as well as the second reference point together with the fourth reference point. Here, too, a correction of a distortion in the evaluation of the indicator device is possible in a comparatively simple manner The above aspects facilitate the evaluation of the indicator device via a (handheld) mobile terminal.

According to a further advantageous aspect, the guide may allow for a translational degree of freedom. The predetermined path and the translational degree of freedom of the guide may be oriented substantially in parallel. An indicator device of this type may, for example, be integrated particularly easily into measuring apparatus with a conventional mechanical indicator and enables particularly quick and error-free data transfer of the determined measured values. Alternatively, the guide may allow for a rotational degree of freedom, the predetermined path being arranged essentially along an arc around an axis of rotation of the rotational degree of freedom.

Alternatively, the predetermined path and the translational degree of freedom of the guide may be oriented at an angle to one another and preferably substantially perpendicular to one another. This aspect makes indicator devices with particularly fine resolution possible.

According to a further advantageous aspect, the first component may have a value indicator window. The value indicator window may extend, at least in sections, along the predetermined path.

According to a further advantageous aspect, the second component may comprise an indicator panel. The indicator panel may have a first value indicator surface and a second value indicator surface. The first value indicator surface may preferably adjoin the second value indicator surface. The first value indicator surface then forms an indicator surface boundary with the second value indicator surface. The indicator surface boundary may preferably be arranged on the indicator panel in such a way that, when the indicator panel is positioned under the value indicator window (shifted along the degree of freedom) by the displacement force when representing a measured value, the indicator surface boundary meets the predetermined path at an angle. If the predetermined path and an edge of the value indicator window coincide locally, the evaluation is facilitated a lot. This aspect allows for the position of the value indicator to be defined by an intersection of the indicator surface boundary with the predetermined path. Contrasting edges, borders and intersections of edges and borders can be evaluated particularly well in the context of digital image recognition.

According to a further advantageous aspect, the first component may comprise a readiness indicator window. The indicator panel may have at least one first readiness indicator surface. In a zero position of the indicator device, the first readiness indicator surface may be arranged under the readiness indicator window. The indicator panel may preferably comprise a second readiness indicator surface. The second readiness indicator surface may be configured and arranged in such a way that it is arranged under the readiness indicator window as soon as the indicator device is not in the zero position. This aspect allows the user to quickly check visually whether the measuring apparatus with the indicator device has been reset to the zero position.

According to a further advantageous aspect, the indicator device may comprise a reset device. The reset device may be configured to bring the second component into a defined zero position via a restoring force.

According to an advantageous aspect, the first component and the second component may be configured such that a displacement force and/or restoring force acting on the second component counteracts a holding force up to a limit value. The second component, or the sled, may preferably comprise a spring tongue. A friction element can be arranged on the spring tongue. The friction element may be pressed against a surface of the first component by the spring tongue, so that a friction force arises as soon as the displacement force or the holding force acts on the second component.

Furthermore, a measuring apparatus for measuring a measured value with the indicator device is provided. In particular, the measuring apparatus may be a peak flow meter.

According to an advantageous aspect, the measuring apparatus may have an air inlet, an air outlet, and an air duct. The air duct may extend from the air inlet to the air outlet.

A displaceable air resistance element may be arranged in the air duct. The air resistance element may be coupled to the second component of the indicator device in such a way that, when the air resistance element is displaced, a displacement force may act on the second component in order to displace the second component.

According to a further advantageous aspect, the air duct may have a lateral opening. The air resistance element may extend through the lateral opening into the air duct. The lateral opening may be sealed by an elastic membrane.

The membrane may advantageously be elastic and have a spring effect. As the displacement of the air resistance element increases, the membrane may reduce the displacement force by an increasing reaction force until a state of equilibrium is established.

According to an advantageous aspect, the measuring apparatus may comprise an outer housing and an inner housing. Preferably, the outer housing may form a hermetically sealed volume together with the inner housing. In particular, the second component may be arranged within the sealed volume. A hermetically sealed volume prevents the ingress of dirt particles and liquids such as saliva.

The outer housing may have a recess for the air inlet, a recess for the air outlet, a recess for the first component of the indicator device, and optionally a recess for a reset device.

The inner housing may form the air duct with the air inlet and the air outlet and contain the lateral opening for the air resistance element.

Particularly advantageously, the recesses in the outer housing, the lateral opening in the inner housing and joints between the outer housing and the inner housing may be hermetically sealed by suitable seals. The above aspects facilitate cleaning the measuring apparatus a lot. The measuring apparatus is therefore far superior to existing solutions in terms of hygiene.

In addition, a computer program product for evaluating the indicator device of a measuring apparatus according to the disclosure is provided. In particular, the evaluation can be carried out by a mobile terminal. The computer program product is configured to carry out the following steps.

According to one aspect, the computer program product is configured to capture a digital image of the indicator device with a camera.

According to a further aspect, the computer program product is configured to evaluate the digital image in order to recognize the reference markers.

According to a further aspect, the computer program product is configured to evaluate the digital image to determine the reference points of the respective reference markers, in particular using edge recognition to recognize the marker surface boundaries.

According to a further aspect, the computer program product may be configured to determine a position and orientation of a predetermined path taking into account the determined reference points.

According to a further aspect, the computer program product may hold value information, wherein the value information assigns a predetermined value to each position along the predetermined path.

According to a further aspect, the computer program product is configured to evaluate the digital image for recognizing and determining the value indicator.

According to a further aspect, the computer program product is configured to determine a position of the value indicator along the predetermined path.

According to an advantageous aspect, the computer program product may be configured to compensate for an optical distortion of the digital image. The distortion may arise, for example, from the central perspective of the camera as soon as the camera is tilted with respect to the indicator device or with respect to the predetermined path. Taking into account at least two geometric pieces of information determined during the evaluation of the digital image; in particular wherein the first geometric piece of information is a diameter, a height or a width of the first reference marker and the second geometric piece of information is correspondingly a diameter, a height or a width of the second reference marker, alternatively the first geometric piece of information is a length and/or an orientation of a certain marker surface boundary of the first reference marker and the second geometric piece of information is a length and/or an orientation of a corresponding marker surface boundary of the second reference marker, alternatively wherein the first geometric piece of information is a distance or a vector between the first reference point and the third reference point and the second geometric piece of information is a distance or a vector between the second reference point and the fourth reference point.

### BRIEF DESCRIPTION OF THE DRAWINGS

Further advantageous aspects are apparent from the following description of preferred exemplary embodiments with reference to the drawings, wherein:

FIG. 1 shows a simplified perspective view of a first exemplary embodiment of a measuring apparatus with an indicator device,

FIG. 2 shows a simplified plan view of some parts of the first component of the indicator device and the indicator panel of the second component of the indicator device,

FIG. 3 shows a simplified plan view of some parts of the first component of the indicator device and the indicator panel of the second component of the indicator device, on the left in the zero position before obtaining the measured value, on the right in a value-indicating position after obtaining the measured value,

FIG. 4 shows a simplified view of different variants of the indicator panel,

FIG. 5 is a simplified exploded view of the measuring apparatus,

FIG. 6 shows a simplified perspective view of an inner housing of the measuring apparatus, including some parts of the second component and the reset device,

FIG. 7 is a simplified sectional view with a horizontal sectional plane through the measuring apparatus,

FIG. 8 shows a simplified sectional view cut horizontally through a variant of the inner housing of FIG. 7,

FIG. 9 shows a simplified sectional view with a vertical sectional plane transversely through the measuring apparatus,

FIG. 10 shows a simplified sectional view with a vertical sectional plane transversely through a variant of the membrane of FIG. 9,

FIG. 11 shows a simplified perspective view of the inner housing with some further component parts,

FIG. 12 shows a simplified plan view of the measuring apparatus with a selective horizontal section through the outer housing to illustrate the displacement of the second component due to the displacement force,

FIG. 13 shows a simplified view of the acquisition and evaluation of a measured value by a mobile terminal device,

FIG. 14 shows a simplified plan view of the measuring apparatus with a selective horizontal section through the outer housing to illustrate the reset of the second component of the indicator device into a zero position via the reset device,

FIG. 15 shows a simplified perspective view of a second exemplary embodiment of a measuring apparatus with an indicator device,

FIG. 16 shows a simplified perspective sectional view with a vertical sectional plane extending along the air duct of the measuring apparatus,

FIG. 17 shows a simplified perspective view of a third exemplary embodiment of a measuring apparatus with an indicator device,

FIG. 18 shows a simplified perspective sectional view of the measuring apparatus with a horizontal sectional plane,

FIG. 19 shows a simplified plan view of a variant of the indicator device 11 with four reference markers, and

FIG. 20 shows a simplified plan view of a further variant of the indicator device 11 with four reference markers.

### DETAILED DESCRIPTION OF EXEMPLARY EMBODIMENTS

In order to avoid unnecessary repetition, the same parts or equivalent parts are provided with the same reference signs, even across different variants of an exemplary embodiment and different exemplary embodiments. In the description, the respective differences are particularly emphasized.

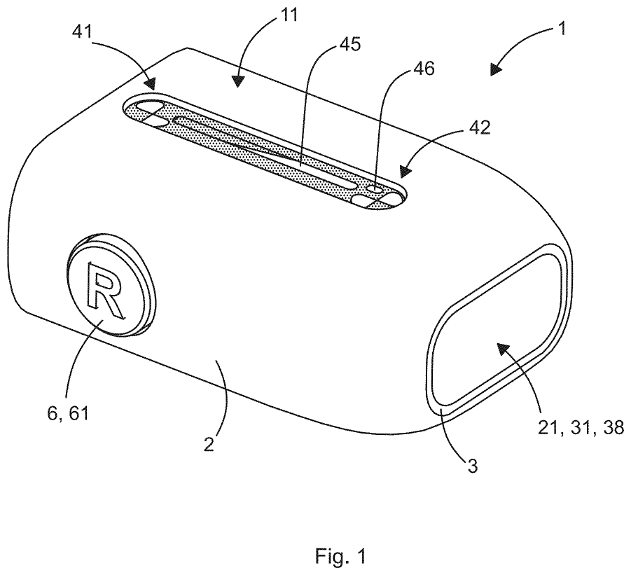

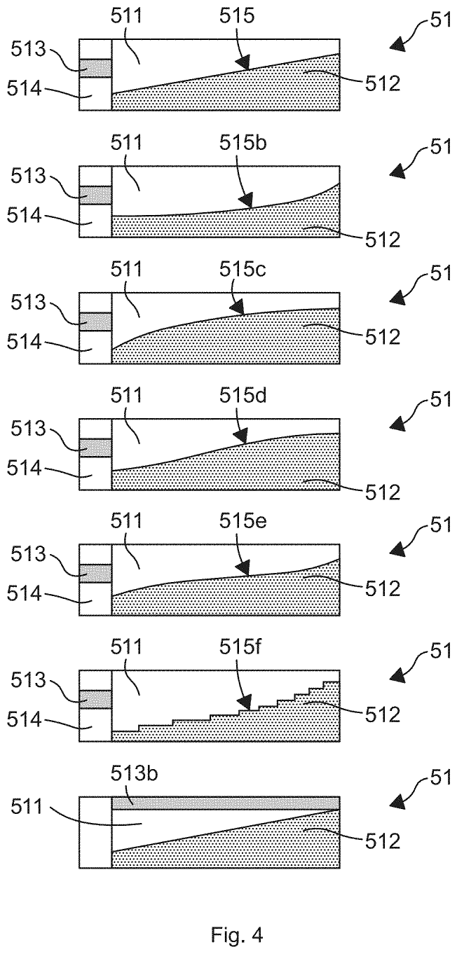

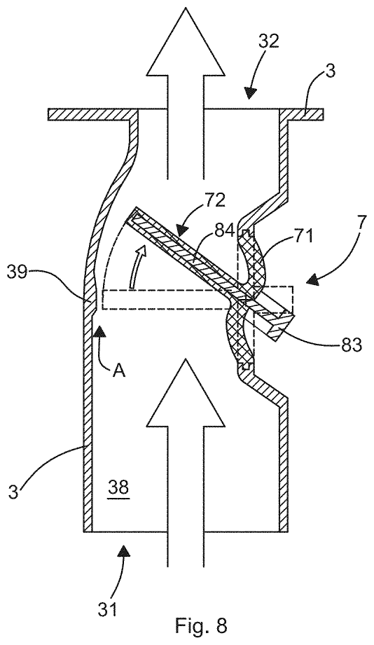

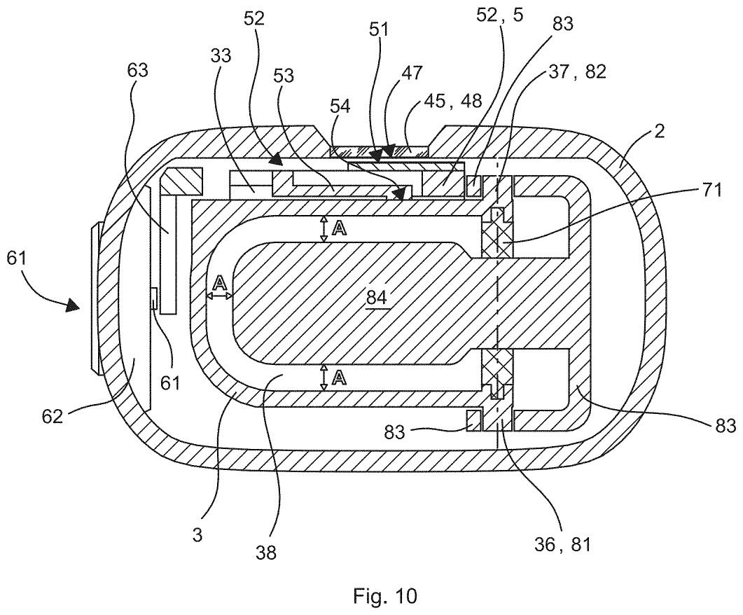

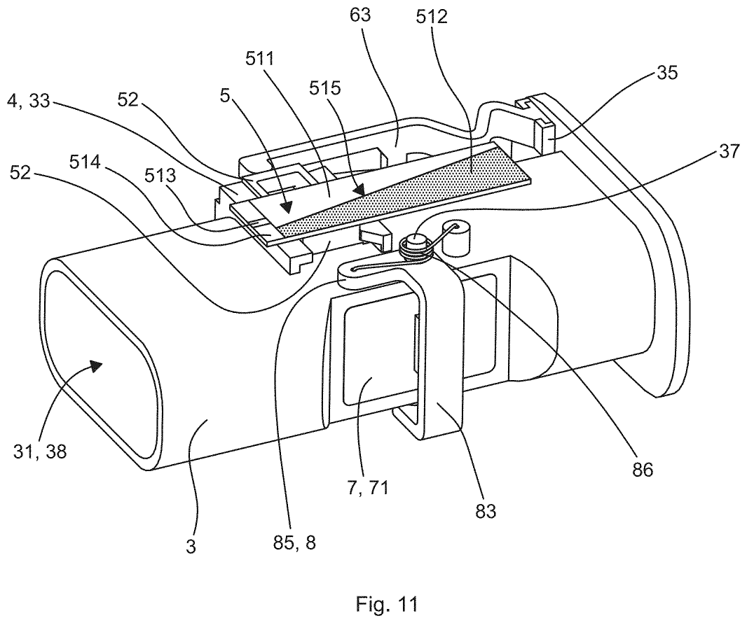

FIGS. 1 to 14 show different views of a first exemplary embodiment of a measuring apparatus. FIG. 4 shows variants of the indicator panel. FIG. 8 shows a variant of the inner housing, FIG. 10 shows a variant of the membrane, and FIG. 11 shows a variant with an additional spring.

FIG. 1 shows the measuring apparatus 1 of the first embodiment in a perspective view. The measuring apparatus 1 is a peak flow meter. The measuring apparatus 1 includes an outer housing 2, an indicator device 11, and a reset button 61 of a reset device 6. The outer housing 2 forms a mouthpiece 21. A recess for an air inlet 31 of the inner housing 3 is provided in the mouthpiece 21. The air inlet 31 is part of the air duct 38.

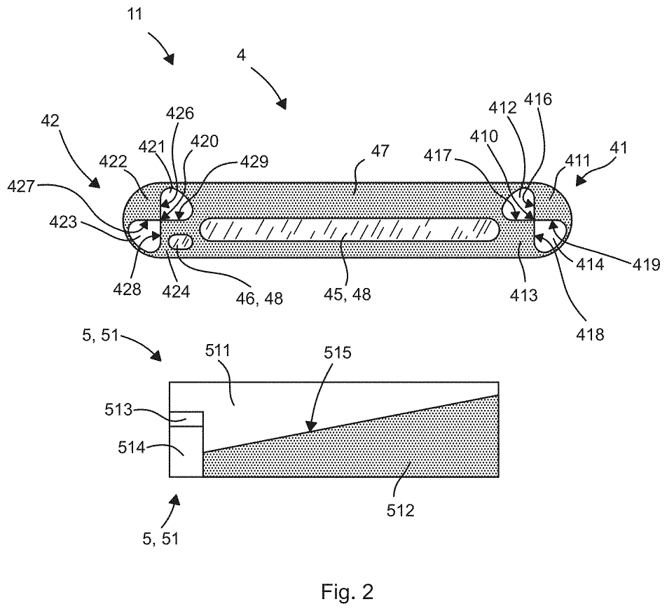

FIG. 2 shows parts of the first component 4 of the indicator device 11 and parts of the second component 5 of the indicator device 11 in a plan view. The first component 4 comprises a machine-readable first reference marker 41 and a machine-readable second reference marker 42.

The first reference marker 41 defines the first reference point 410 via the first marker surface 411, the second marker surface 412, the third marker surface 413 and the fourth marker surface 414. Each of the four marker surfaces 411-414 adjoins the other marker surfaces of the first reference marker 41 at the reference point 410. The first and third marker surfaces 411, 413 are black, the second and fourth marker surfaces 412, 414 are white. The selection of other colors is possible. A strong contrast between the respective colors is advantageous. In this way, easily recognizable contrast edges are formed at the marker surface boundaries between the respective adjacent marker surfaces 411-414. The marker boundary surfaces meet in the center of the reference marker 41 and define the reference point 410.

The second reference marker 42 is configured in a manner analogous to the first reference marker 41. The second reference marker 42 defines the second reference point 420 via the first marker surface 421, the second marker surface 422, the third marker surface 423 and the fourth marker surface 424. Each of the four marker surfaces 421-424 adjoins the other marker surfaces of the second reference marker 42 at the reference point 420. The first and third marker surfaces 421, 423 are white, the second and fourth marker surfaces 422, 424 are black. The second reference marker 42 is thus rotated by 90 degrees with respect to the first reference marker 41. The selection of other colors instead of black and white is possible. A high (clearly visible) contrast between the respective colors is advantageous. The marker boundary surfaces meet in the center of the reference marker 42 and define the reference point 420.

Further reference markers, in particular a third and fourth reference marker, may be constructed in an analogous manner without need for repeated description.

The position and orientation of a predetermined path are defined by the first reference point 410 and the second reference point 420. In the first exemplary embodiment, the predetermined path extends in a straight line from the first reference point 410 to the second reference point 420.

Positions along the predetermined path 13 are associated with measured values, for example via a corresponding table of values.

Directly adjoining the predetermined path and along the predetermined path 13, a first viewing window 45 extends in the first component 4. The first viewing window 45 is used to indicate the value. One edge of the viewing window 45 coincides locally with the predetermined path 13.

The first component 4 also includes a second viewing window 46. The second viewing window 46 is used to indicate readiness.

The first viewing window 45 and the second viewing window 46 are hermetically sealed by a transparent material, for example a transparent plastic. A non-transparent mask is applied to parts of the transparent material 48.

The second component 5 comprises the indicator panel 51. The second component 5 is arranged below the first component. The indicator panel 51 includes a first value indicator surface 511 (shown in white) and a second value indicator surface 512 (shown in black). The first value indicator surface 511 and the second value indicator surface 512 adjoin one another and thereby form an indicator boundary line 515.

The indicator panel 51 includes a first readiness indicator surface 513 and a second readiness indicator surface 514. The first readiness indicator surface 513 is preferably green. The second readiness indicator surface 514 is preferably red.

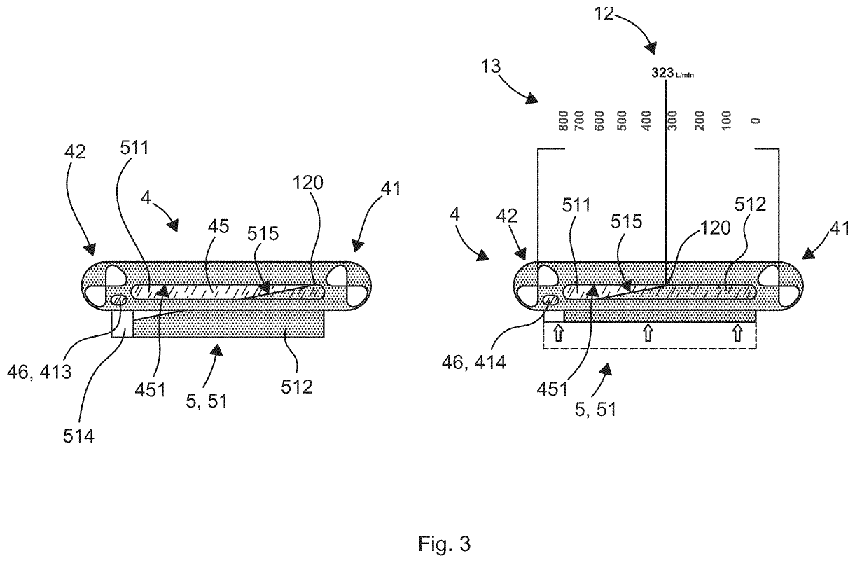

FIG. 3 shows, on the left-hand side, parts of the first component 4 and parts of the second component 5 in a zero position, both in plan view. The first component 4 at least partially obscures the second component 5. The indicator boundary line 515 intersects the edge of the first viewing window 45 extending along the predetermined path in a zero position. The first readiness indicator surface 513 is located directly below the second viewing window 46 and thus signals that the indicator device 11 has been reset.

On the right-hand side of FIG. 3, a measured value is represented by the relative position of the movable second component 5 with respect to the first component 4. For better visibility, the predetermined path 13 is shifted out of its actual position defined by the reference markers 41, 42. The intersection of the window edge 451 of the first viewing window 45 located on the predetermined path 13 and the indicator boundary line 515 defines the reference point 120 of the value indicator 12. The value indicator 12 is formed by the first value indicator surface 512, the second value indicator surface 513, and the mask 47.

The position of the value indicator 12 along the predetermined path may be converted into a measured value, here 323 L/min. For this purpose, a known value is assigned to each position along the predetermined path 13.

FIG. 4 shows different variants of the indicator panel 51, each in a top view. In the case of the indicator device 11 according to the first exemplary embodiment, a first displacement of the indicator panel along the guide 33 leads to a second displacement of the intersection of the indicator boundary line 515-515f with the predetermined path 13. T (local) slope of the indicator boundary line 515-515f can be used to adjust the ratio of the first displacement to the second displacement. A comparatively greater slope of the indicator boundary line 515-515f thus results in a lower sensitivity (resolution) of the indicator device 11.

The first indicator boundary line 515 is a straight line with a constant slope. With a slope of approximately 0.3, the sensitivity is approximately 3. In other words, a displacement of the indicator panel 51 by 1 mm along the guide results in a displacement of the value indicator along the predetermined path by approximately 3 mm.

The second indicator boundary line 515b has an essentially exponential function, the slope increases to the right. This means that the sensitivity is rather low in a lower value range and increases with increasing values. Such an indicator boundary line would be suitable, for example, for athletes with a rather good pulmonary function.

The third indicator boundary line 515c has an essentially logarithmic function. This means that the sensitivity is rather high in a lower value range and decreases with increasing values. Such an indicator boundary line would be suitable for COPD patients, for example.

The fourth indicator boundary line 515d is essentially S-shaped. This means that the sensitivity is high at the start and end of the value scale and decreases in a medium range.

The fifth indicator boundary line 515e is essentially ˜-shaped. This means that the sensitivity in the peripheral areas of the value scales is rather low and increases towards the medium range.

The sixth indicator boundary line 515f has steps. A stepped indicator boundary line results in discrete value jumps.

The lowermost indicator panel 51 has an alternative first readiness indicator surface 513b. In the form shown, the function of the second viewing window 46 can be assumed by the first viewing window 45. In a zero position of the second component 5, the first readiness indicator surface 513b is visible in the first viewing window 45.

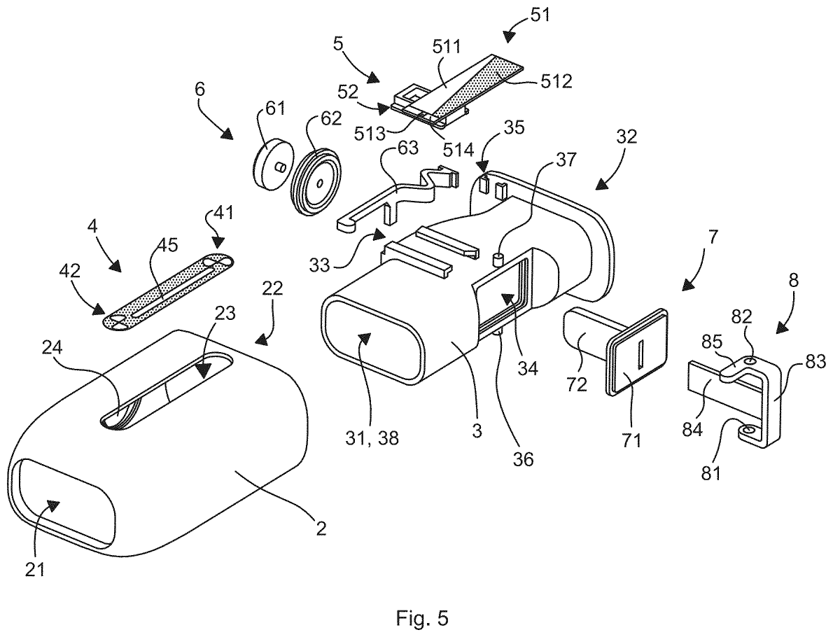

FIG. 5 shows an exploded view of the measuring apparatus 1. The measuring apparatus 1 is a peak flow meter.

The outer housing 2 has a mouthpiece in which a recess 21 for the air inlet of the inner housing 3 is formed. The outer housing 2 furthermore has a recess 22 for the air outlet, a recess 23 for the visible part of the first component 4, and a recess 24 for a reset button 61 of a reset device 6.

The outer housing 2 can be made of a transparent plastic (at least in sections). In this case, the recess 23 for the first component 4 can be omitted.

The inner housing 3 includes an air duct 38 with an air inlet 31 and an air outlet 32. The inner housing has an opening 34 for a membrane 7 on the side. A guide 33 for the second component 5 of the indicator device 11, a holder 35 for the reset lever 63 of the reset device 6, and bearing points 36, 37 for the displacement unit 8 are arranged on the outside of the inner housing 3. The bearing points 36, 37 are positioned such that an axis of rotation is formed in the membrane 71.

The measuring apparatus comprises a reset device 6 with a reset button 6 movably supported by a sealing disk 62. The sealing disk hermetically seals the associated recess 24. When the reset button 6 is pressed, the reset lever 63 is deflected and presses the second component 5 along the guide 33 back to the zero position.

The membrane 7, 71 hermetically seals the associated opening 34. The membrane 7 also includes an air resistance element 72. The air resistance element 72 extends transversely to the air duct 38 through the opening 34 and into the air duct 38, so that the air resistance element 72 closes a large part of the cross section of the air duct 38 and only leaves a gap A around the air resistance element 72. An insert 84 of the displacement unit 8 is inserted into the air resistance element 72 and stiffens the air resistance element 72.

The displacement unit 8 comprises a C-shaped web 83 with bearing points 81, 82 which correspond to the bearing points 36, 37 on the inner housing 3. A slide 85 protrudes from the upper bearing point 37 at right angles to the web 83. The insert 84 extends from the web 83 in the direction of the axis of rotation.

Due to the C-shaped structure, the axis of rotation of the bearing points 81, 82 lies both in the plane of the membrane 71 and in the plane of the air resistance element 72 extending transversely to the membrane 71.

The outer housing 2 and the inner housing 3 form a hermetically sealed volume. Appropriate seals are provided in the recesses 23, 24, 34 and joints. In this context, a hermetic seal is understood to mean any seal that prevents the ingress of liquids and particles under the operating, cleaning and storage conditions typical for the product. The hermetic seal is advantageously also gas-tight under the conditions mentioned.

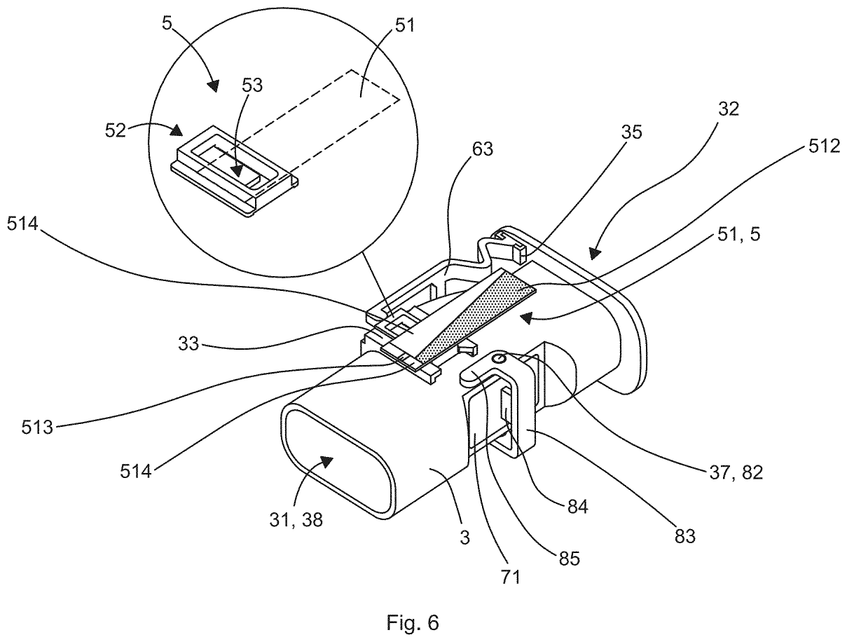

FIG. 6 shows the assembled measuring apparatus 1 without an outer housing in a perspective view. The second component 5 is shown in a magnification. The second component 5 comprises the indicator panel 51, a sled 52 guided within the guide along a translational degree of freedom, and a spring tongue 53 with a friction element 54.

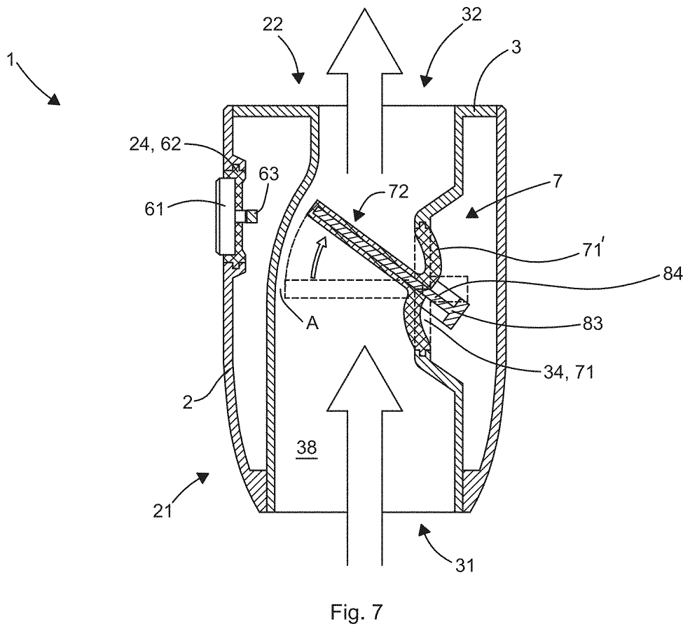

FIG. 7 shows a section through the measuring apparatus 1 in a horizontal plane. In a superimposed illustration, the air resistance element 72 is shown both in a rest position and in a position displaced by an air flow. The displacement of the air resistance element 72 tensions the elastic membrane 71, so that a restoring force arises in the membrane 71. The restoring force counteracts the displacement by the air flow. The wall of the air duct 38 is configured such that a defined free cross section A is formed between the wall of the air duct and the resistance element even in a displaced position of the air resistance element 72.

The sealing membrane 7 contains an elastic material, preferably an elastic silicone-like material with a long service life. The membrane 7 has two functions. On the one hand the sealing function: The membrane 7 hermetically seals the side opening 34 in the air duct. Furthermore, the membrane 7 has a defined tension force and thus assumes the function of the spring 86.

The air flow hits the movable air resistance element (wing) 72 in the working chamber (defined by the air duct 38), thereby displacing it in the air flow direction relative to the axis of rotation. The sealing membrane 71 is thereby deformed. Due to the elastic properties of the sealing membrane 71, a restoring force arises in the membrane 71, which resists the air pulse and tries to move the movable air resistance element 72 into the original position. The original position of the movable air resistance element 72 is indicated by the dashed line.

At the end of the breathing maneuver and as soon as the restoring force of the movable air resistance element 72 is greater than the force of the air pulse in the working chamber, the peak flow value is reached. In this way, the degree of displacement of the resistance element 72 is proportional to the force of the maximum air flow generated during forced expiration. The movable air resistance element 72 springs back to the original position.

Since the peak flow value is measured in liters per minute in a measuring range between 0 L/min and 800 L/min, the flexibility or tension force of the sealing membrane 71 should be configured such that it corresponds to the peak flow value and does not exceed the 800 L/min mark.

As an alternative or in addition, a spring 86 may be added to the sealing membrane 71 in order to achieve the desired spring properties overall.

FIG. 8 shows a variant of the inner housing 3. The variant is particularly suitable for COPD patients, for example. The inner housing 3 of the variant differs from the first exemplary embodiment by an additional tapering of the free cross section A in the area of the air resistance 72 in the rest position. As a result of the tapering, the proportion of the air flow that can freely flow past the air resistance element 72 is reduced. This increases the displacement of the air resistance element 72 and accordingly also of the displacement unit 8 in the case of small air flows. The sensitivity/resolution of the measuring apparatus is increased in the low value range.

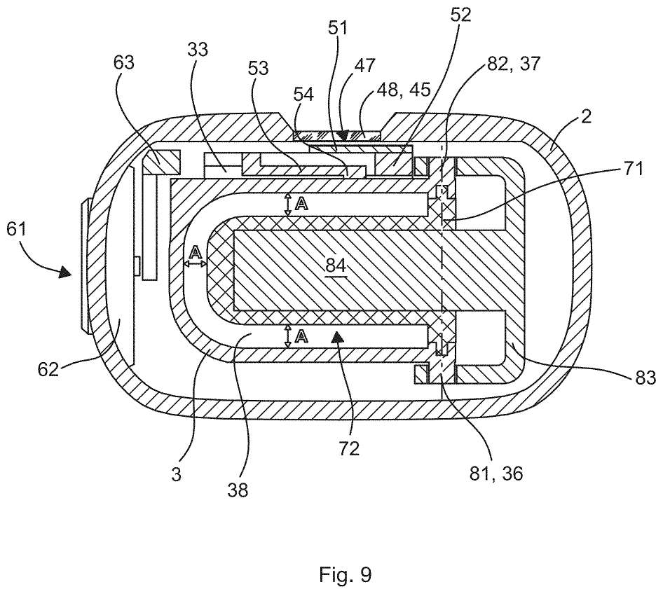

FIG. 9 shows the measuring apparatus 1 in a sectional view with a vertical sectional plane extending transversely through the air duct 38 and longitudinally through the insert 84. In this illustration, the spring tongue 53 and the friction element 54 can be seen particularly well.

The movable air resistance element 72 has a defined surface and shape. The air resistance element 72 has the function of absorbing the force of the air pulse and translating it into motion. The gap A between the inner walls of the air duct 38 and the end of the air resistance element 72 is selected such that the maximum flow resistance in the air duct 38 is not exceeded.

A mask 47 is applied to the transparent material 48 of the first component 4 at least in partial areas on a bottom side.

FIG. 10 shows a variant of the membrane 7 and the insert 84. In this, the air resistance element is not formed by a part of the membrane 7, but by an enlarged insert 84. In order to avoid the formation of a gap even when the membrane 7 is deflected, the membrane 7 and the insert 84 are preferably joined in a materially bonded manner, that is, in particular, glued or welded.

FIG. 11 shows another variant of the first exemplary embodiment. The variant comprises a torsion spring 86 fastened to the displacement unit 8 in the area of the slide 85 with a first leg. A second leg of the torsion spring 86 is held on the inner housing 3 by a holder. The torsion spring 86, like the membrane 7, generates a counterforce counteracting the displacement of the air resistance element 72.

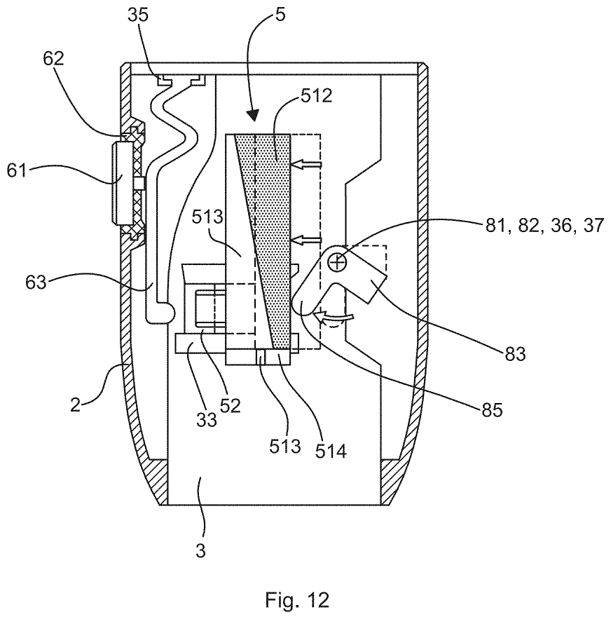

FIG. 12 shows the displacement of the second component 5 by the displacement unit. The displacement unit 8 rotates about its axis of rotation as a result of the displacement of the air resistance element 72. The slide 85 of the displacement unit 8 moves the second component 5 along a translational degree of freedom of the guide 33 as the rotation progresses.

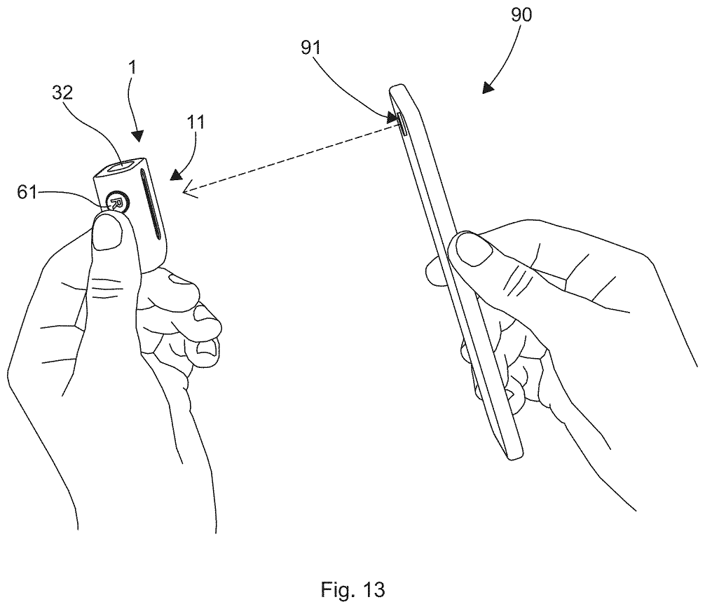

FIG. 13 shows the evaluation of the value indicated by the indicator device 11. A mobile terminal 90 with a camera 91 is used for this purpose. The mobile terminal 90 includes a corresponding computer program product (an app). The computer program product is configured to carry out the following steps:

Capturing a digital image of the indicator device 11 with a camera 91.

Evaluating the digital image to identify the reference markers 41-44.

Evaluating the digital image to determine the reference points 410-440 of the respective reference markers 41-44.

Determining the position and orientation of a predetermined path 13 taking into account the determined reference points 410-440.

Evaluating the digital image to recognize and determine the value indicator 12.

Determining a position of the value indicator 12 along the predetermined path 13.

Optionally, the computer program product may also be configured to correct a distortion of the digital image. For compensation, the computer program product uses at least two additional pieces of geometric information that it extracts from the digital image of the reference markers.

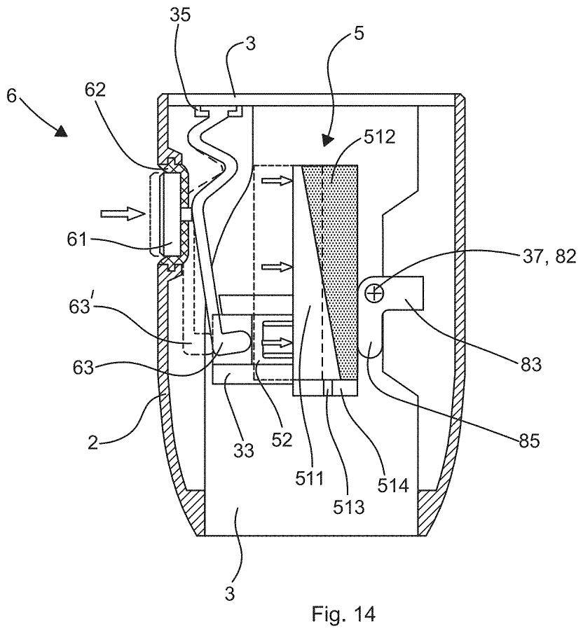

FIG. 14 shows a plan view of the measuring apparatus 1. The outer housing 3 is sectioned at the level of the reset button 61 in a horizontal sectional plane. When resetting the second component 5 of the indicator device 11, the operator presses the reset button 61, whereby the reset button 61 deflects the reset lever 63 such that one bent end thereof pushes the second component back into the zero position.

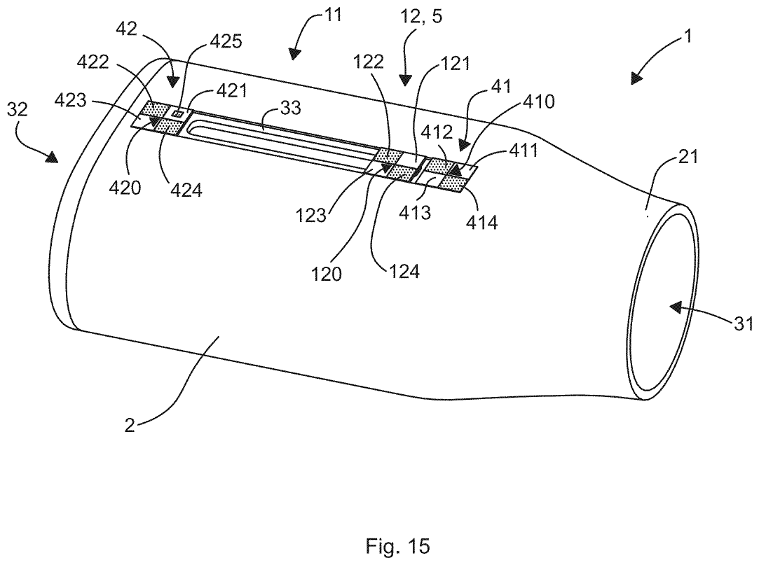

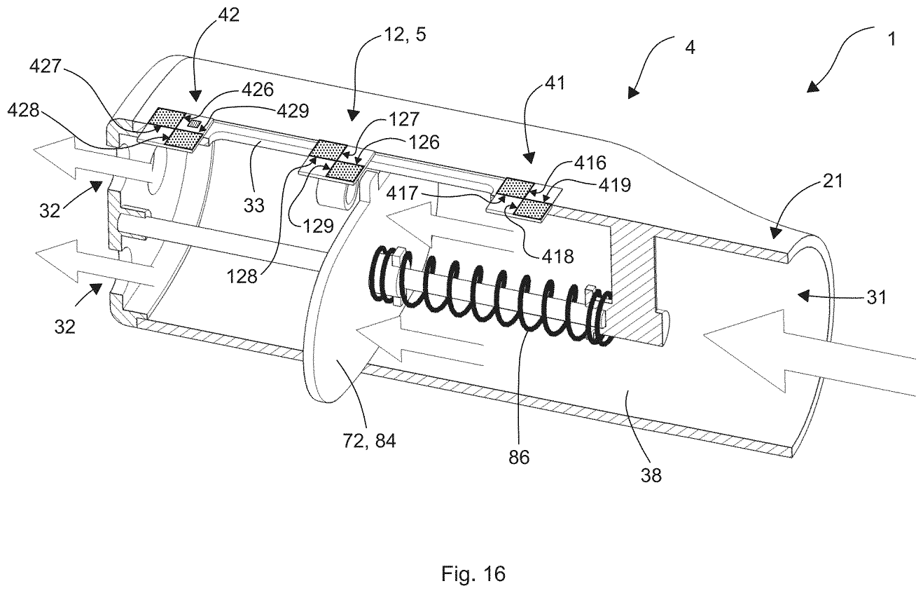

FIGS. 15 and 16 show a second exemplary embodiment of the measuring apparatus 1.

The reference markers 41, 42 are configured in a manner comparable to the first exemplary embodiment. In contrast to the first exemplary embodiment, the fixed reference markers 41, 42 have a square outer shape instead of the round outer shape. The second reference marker 42 is not rotated with respect to the first reference marker 41. Instead, the second reference marker 42 has an additional marking 425 in order to be able to distinguish the second reference marker 42 from the first reference marker 41.

The value indicator 12 is movably held in a guide 33 with a translational degree of freedom. The design of the value indicator 12 corresponds to the first reference marker 41. The evaluation of the indicator device substantially corresponds to the evaluation of the first exemplary embodiment.

In FIG. 16, the second exemplary embodiment is shown as a sectional view with a vertical sectional plane extending along the air duct 38 from the air inlet 31 to the air outlet 32. The air resistance element 72 slides translationally along a guide rod when displaced by an air flow. With increasing displacement, a spring 86 builds up a counterforce which pulls the air resistance element 72 back into a rest position when the air flow decreases. The air resistance element 72 includes a driver which takes the value indicator 12 along up to a maximum value. A defined friction force prevents the unintentional displacement of the value indicator 12.

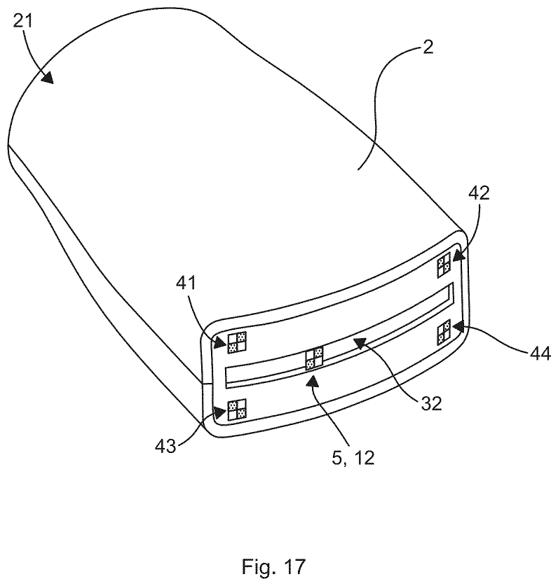

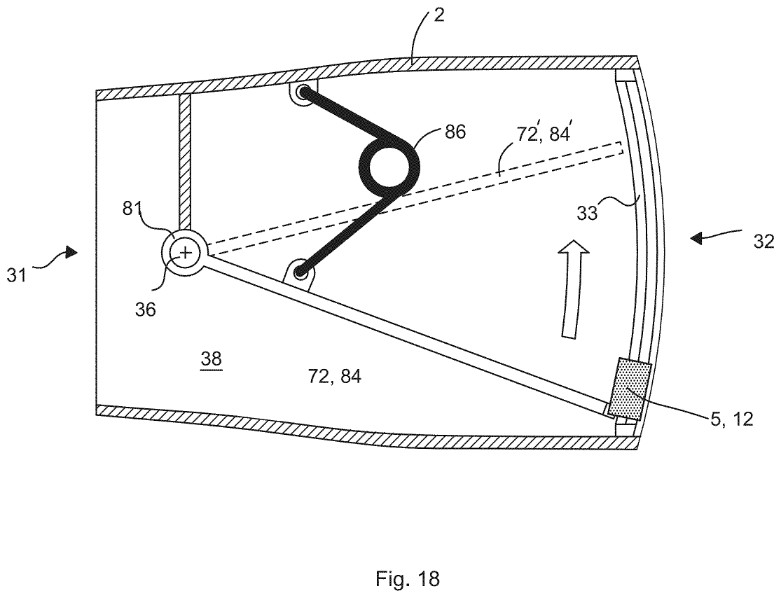

FIGS. 17 and 18 show a third embodiment of the measuring apparatus 1, one in a perspective view and one in a sectional view with a horizontal sectional plane.

The measuring apparatus 1 includes a housing with an air inlet 31 and an air outlet 32. The air resistance element 72 is rotatably mounted at a bearing point 36. A spring 86 is mounted on the housing 3 with a first end and on the air resistance element 72 with a second end.

The air resistance element 72 closes the air duct almost completely in a base position. An air flow displaces the air resistance element 72 against the spring force of the spring 86 until a maximum displacement is established at an equilibrium point. Up to the maximum displacement, the air resistance element 72 takes the value indicator 12 along with it on a second component 5. The second component 5 is movable along a translational degree of freedom of a guide 33.

The measuring apparatus 1 includes four reference markers 41-44 arranged on both sides at the outer ends of the predetermined path. The design of the value indicator 12 corresponds to the reference markers 41-44. The second reference marker 42 is rotated by 90 degrees with respect to the first reference marker 41.

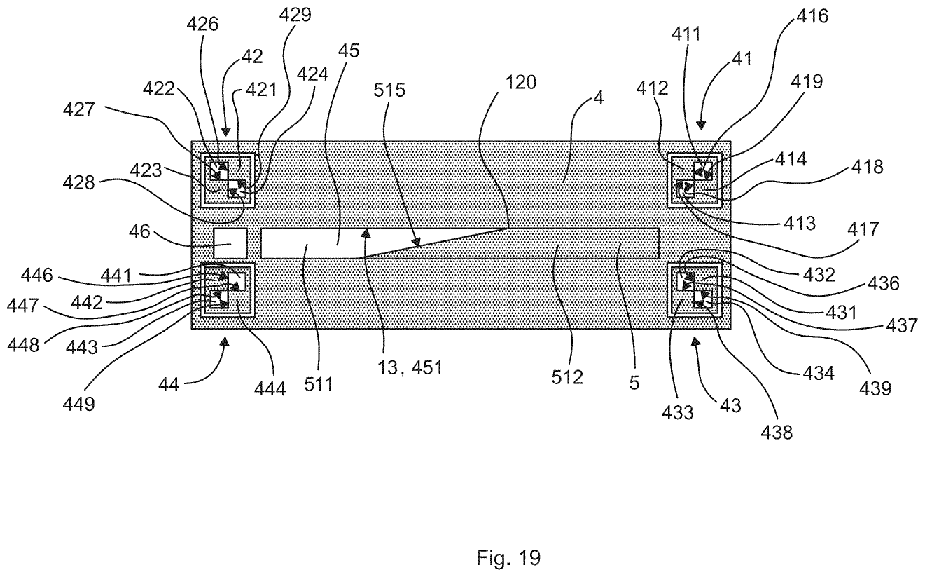

FIG. 19 shows a variant of the indicator device 11 of the first exemplary embodiment, but with four reference markers 41-44. The third and fourth reference markers 43, 44 are configured corresponding to the first and second reference markers 41, 42. The second reference marker 42 is configured corresponding to the first reference marker. The second reference marker 42 is rotated by 90 degrees with respect to the first reference marker 41. The third and fourth reference markers 43, 44 are rotated by 90 degrees with respect to the first and second reference markers 41, 42.

The reference markers 41-44 each have four marker surfaces 411-414, 421-414, 431-433, 441-444. The marker surfaces of the respective reference marker 41-44 define an associated reference point 410, 420, 120.

The first marker surface 411 of the first reference marker 41 adjoins the second marker surface 412 and the fourth marker surface 414. The second marker surface 412 of the first reference marker 41 adjoins the third marker surface 413 and the first marker surface 411. The third marker surface 413 of the first reference marker 41 adjoins the fourth marker surface 414 and the second marker surface 412. The fourth marker surface 414 of the first reference marker 41 adjoins the first marker surface 411 and the third marker surface 413.

The first and second marker surfaces 411, 412 form a first marker surface boundary 416. The second and third marker surfaces 412, 413 form a second marker surface boundary 417. The third and fourth marker surfaces 413, 414 form a third marker surface boundary 418. The fourth and first marker surfaces 414, 411 form a fourth marker surface boundary 419.

The associated first reference point 410 is defined by a common point of intersection of at least two marker surface boundaries 416-419, here all marker surface boundaries 416-419.

A machine-readable third reference marker 43 with a third reference point 430 is associated with the first reference marker 41. A machine-readable fourth reference marker 44 with a fourth reference point 440 is associated with the second reference marker 42. The position and orientation of the predetermined path 13 are defined by the first reference point 410 together with the third reference point 430 as well as with the second reference point 420 together with the fourth reference point 440.

The third and fourth reference markers are rotated by 90 degrees with respect to the first and second reference markers. The rotation is optional.

The value indicator 12 has first and second marker surfaces 511, 512/47. The marker surfaces 411, 412/47 of the value indicator 12 define the reference point 120.

The first marker surface 511 adjoins the second marker surface 512/47. Similarly, the second marker surface 512/47 adjoins the first marker surface 511.

A first marker surface boundary 515 is formed between the first marker surface 511 and the second marker surface 512. In other words, the indicator boundary line 515 is formed between the first value indicator surface 511 and the second value indicator surface 512.

A second marker surface boundary 451 is formed between the second marker surface 47 and the first marker surface 515. In other words, the window edge 451 is located between the mask 47 and the first value indicator surface 511.

The reference point 120 of the value indicator 12 is defined by the intersection of the first marker surface boundary 515 and the second marker surface boundary 451. In other words, the reference point 120 of the value indicator 12 is defined by the intersection of the indicator surface boundary 515 and the window edge 451.

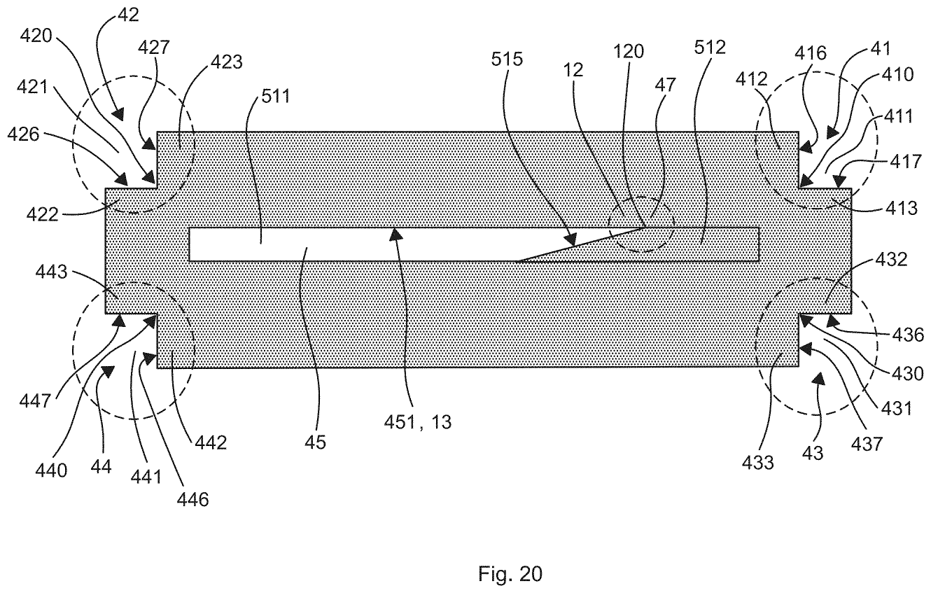

FIG. 20 shows a simplified plan view of a further variant of the indicator device 11 with four reference markers 41-44. In principle, two reference markers 41, 42 are sufficient.

A third reference marker 43 and possibly a fourth reference marker 44, however, simplifies the evaluation of the digital image of the indicator device 11 recorded by a camera 91 and improves the measurement result. The digital image is distorted by (unintentional) tilting between the indicator device 11 and the camera 91 during the capture. The distortion can be corrected by an appropriate compensation. This requires (geometric) information that can be used to determine the degree of the distortion.

For example, the degree of the distortion can be inferred by taking into account at least two pieces of geometric information determined during the evaluation of the digital image.

In the present case, the distance or vector between the first reference marker 41 and the third reference marker 43 and the distance or vector between the second reference marker 42 and the fourth reference marker 44 can be determined and used for correcting the digital image.

Alternatively, the vector between the first reference marker 41 and the second reference marker 42 and the vector between the first reference marker 41 and the third reference marker 43 can be evaluated.

Alternatively, the first geometrical piece of information can be a diameter, a height or a width of the first reference marker 41 and, correspondingly, the second geometrical piece of information can be a diameter, a height or a width of the second reference marker 42. Then, the third and fourth reference markers 43, 44 are not required.

Alternatively, the first geometric piece of information can be a length and/or an orientation of a specific marker surface boundary 416-419 of the first reference marker 41 and the second geometric piece of information can be a length and/or an orientation of a corresponding marker surface boundary 426-429 of the second reference marker 42.

The first reference marker 41 has first and second marker surfaces 411, 412. The marker surfaces 411, 412 of the first reference marker 41 define the first reference point 410.

The first marker surface 411 adjoins the second marker surface 412. Similarly, the second marker surface 412 adjoins the first marker surface 411. A first marker surface boundary 416 and a second marker surface boundary 417 are formed between the first marker surface 411 and the second marker surface 412. The first reference point 410 is defined by the intersection of the first marker surface boundary 416 and the second marker surface boundary 417.

The second reference marker 42 has first and second marker surfaces 421, 422. The marker surfaces 421, 422 of the second reference marker 42 define the second reference point 420.

The first marker surface 421 adjoins the second marker surface 422. Similarly, the second marker surface 422 adjoins the first marker surface 421. A first marker surface boundary 426 and a second marker surface boundary 427 are formed between the first marker surface 421 and the second marker surface 422. The second reference point 420 is defined by the intersection of the first marker surface boundary 426 and the second marker surface boundary 427.

Advantageously, a machine-readable fourth reference marker 44 with a fourth reference point 440 is associated with the second reference marker 42. The position and orientation of the predetermined path 13 are defined by the first reference point 410 together with the third reference point 430 and by the second reference point 420 together with the fourth reference point 440.

The third reference marker 43 has first and second marker surfaces 431, 432. The marker surfaces 431, 432 of the second reference marker 43 define the third reference point 430.

The first marker surface 431 adjoins the second marker surface 432. Similarly, the second marker surface 432 adjoins the first marker surface 431. A first marker surface boundary 436 and a second marker surface boundary 437 are formed between the first marker surface 431 and the second marker surface 432. The second reference point 430 is defined by the intersection of the first marker surface boundary 436 and the second marker surface boundary 437.

The fourth reference marker 44 has first and second marker surfaces 441, 442. The marker surfaces 441, 442 of the fourth reference marker 44 define the fourth reference point 440.

The first marker surface 441 adjoins the second marker surface 442. Similarly, the second marker surface 442 adjoins the first marker surface 441. A first marker surface boundary 446 and a second marker surface boundary 447 are formed between the first marker surface 441 and the second marker surface 442. The fourth reference point 440 is defined by the intersection of the first marker surface boundary 446 and the second marker surface boundary 447.

A machine-readable third reference marker 43 with a third reference point 430 is associated with the first reference marker 41. The position and orientation of the predetermined path 13 are defined by the first reference point 410 together with the third reference point 430 and the second reference point 420.

The value indicator 12 has first and second marker surfaces 511, 512/47. The marker surfaces 411, 412/47 of the value indicator 12 define the reference point 120.

The first marker surface 511 adjoins the second marker surface 512/47. Similarly, the second marker surface 512/47 adjoins the first marker surface 511.

A first marker surface boundary 515 is formed between the first marker surface 511 and the second marker surface 512. In other words, the indicator boundary line 515 is formed between the first value indicator surface 511 and the second value indicator surface 512.

A second marker surface boundary 451 is formed between the second marker surface 47 and the first marker surface 515. In other words, the window edge 451 is located between the mask 47 and the first value indicator surface 511.

The reference point 120 of the value indicator 12 is defined by the intersection of the first marker surface boundary 515 and the second marker surface boundary 451. In other words, the reference point 120 of the value indicator 12 is defined by the intersection of the indicator surface boundary 515 and the window edge 451.

Although the disclosure has been described hereinabove with reference to specific embodiments, it is not limited to these embodiments and no doubt further alternatives will occur to the skilled person that lie within the scope of the disclosure as claimed.

1. An indicator device for representing a measured value, said indicator device comprising:
a first component and a second component;
said second component being movable with respect to the first component, said first component including a machine-readable first reference marker, a machine-readable second reference marker spaced from the machine-readable first reference marker, and a guide for the second component;
wherein the machine-readable first reference marker defines a first reference point and the machine-readable second reference marker defines a second reference point, and wherein a position and orientation of a predetermined path are defined by the machine-readable first reference point and the machine-readable second reference point;
wherein the second component is positionable along the guide by a displacement force; and
wherein the second component alone, or along with the first component, forms a machine-readable value indicator, a position of the machine-readable value indicator along the predetermined path representing the measured value.

2. The indicator device according to claim 1, wherein the machine-readable first reference marker, the machine-readable second reference marker, and/or the machine-readable value indicator each have at least two marker surfaces, or have four marker surfaces, and wherein the marker surfaces of the respective machine-readable first and/or second reference marker or machine-readable value indicator define a reference point.

3. The indicator device according to claim 2, wherein at least two of the marker surfaces of the respective machine-readable first an/or second reference marker or machine value indicator each adjoin at least one other of the marker surfaces, a marker surface boundary being formed between two adjoining marker surfaces, and wherein the associated reference point is defined by a common intersection of at least two marker surface boundaries.

4. The indicator device according to one of claim 1, wherein a machine-readable third reference marker with a third reference point is associated with the machine-readable first reference marker, and wherein the position and orientation of the predetermined path are defined by the first reference point together with the third reference point and the second reference point, optionally wherein a machine-readable fourth reference marker with a fourth reference point is associated with the machine-readable second reference marker, wherein in particular the position and orientation of the predetermined path is defined by the first reference point together with the third reference point and by the second reference point together with the fourth reference point.

5. The indicator device according to one of claim 1, wherein the guide allows for a translational degree of freedom, and wherein the predetermined path and the translational degree of freedom of the guide are oriented substantially in parallel, alternatively wherein the guide allows for a rotational degree of freedom and wherein the predetermined path is arranged substantially along an arc around an axis of rotation of the rotational degree of freedom.

6. The indicator device according to one of claim 1, wherein the guide allows for a translational degree of freedom and wherein the predetermined path and the translational degree of freedom of the guide are at an angle, in particular substantially perpendicular to each other.

7. The indicator device according to claim 6, wherein the first component comprises a value indicator window, the value indicator window extending along the predetermined path at least in sections, wherein the second component comprises a indicator panel, the indicator panel having a first value indicator surface and a second value indicator surface, and wherein the first value indicator surface adjoins the second value indicator surface and forms an indicator surface boundary with the second value indicator surface in such a way that, when the indicator panel was positioned under the value indicator window by the displacement force during representation of a measured value, the indicator surface boundary meets the predetermined path and in particular an edge of the value indicator window at an angle, and wherein the position of the value indicator is defined by an intersection of the indicator surface boundary with the predetermined path.

8. The indicator device according to claim 7, wherein the first component comprises a readiness indicator window, wherein the indicator panel has at least one first readiness indicator surface, wherein, in a zero position of the indicator device, the first readiness indicator surface is arranged under the readiness indicator window, in particular wherein the indicator panel includes a second readiness indicator surface, the second readiness indicator surface being formed and arranged such that the second readiness indicator surface is arranged under the readiness indicator window when the indicator device is not in the zero position.

9. The indicator device according to claim 1, wherein the indicator device comprises a reset device configured to bring the second component into a zero position via a restoring force.

10. The indicator device according to claim 1, wherein the first component and the second component are configured such that a displacement force and/or restoring force acting on the second component counteracts a holding force.

11. A measuring apparatus comprising the indicator device according claim 1, in particular wherein the measuring apparatus is a peak flow meter.

12. The measuring apparatus according to claim 11, wherein the measuring apparatus comprises an air inlet, an air outlet, and an air duct, the air duct extending from the air inlet to the air outlet, wherein a displaceable air resistance element is arranged in the air duct, and wherein the air resistance element is coupled with the second component of the indicator device in such a manner that, when the air resistance element is displaced, a displacement force can act on the second component in order to displace the second component.

13. The measuring apparatus according to claim 12, wherein the air duct has a lateral opening, the air resistance element extending through the lateral opening into the air duct, and wherein the lateral opening is sealed by an elastic membrane, in particular wherein the elastic reduces the displacement force by an increasing reaction force with increasing displacement of the air resistance element.

14. The measuring apparatus according to claim 13, wherein the measuring apparatus comprises an outer housing and an inner housing, wherein the outer housing, together with the inner housing, forms a hermetically sealed volume, and wherein the second component is arranged within the sealed volume; in particular wherein the outer housing has a recess for the air inlet, a recess for the air outlet, a recess for the first component of the indicator device, and optionally a recess for a reset device; in particular wherein the inner housing forms the air duct with the air inlet and the air outlet and includes the lateral opening for the air resistance element; in particular wherein the recesses in the outer housing, the lateral opening in the inner housing, and joints between the outer housing and the inner housing are hermetically sealed by seals.

15. A computer program product for evaluating the indicator device of the measuring apparatus according to claim 11, in particular via a mobile terminal, configured to carry out the steps of:
a) capturing a digital image of the indicator device with a camera,
b) evaluating the digital image to recognize the machine-readable first and second reference markers, in particular using edge recognition to recognize marker surface boundaries,
c) evaluating the digital image to determine the reference points of the respective machine-readable first and second reference markers,
d) determining the position and orientation of the predetermined path taking into account the determined first and second reference points,
e) evaluating the digital image to recognize and determine the machine-readable value indicator,
f) determining a position of the machine-readable value indicator along the predetermined path, in particular wherein the computer program product is further configured to carry out an optional step of:
g) correcting an optical distortion of the digital image taking into account at least first and second geometric pieces of information determined during evaluation of the digital image; in particular wherein the first geometric piece of information is a diameter, a height, or a width of the machine-readable first reference marker and correspondingly the second geometric piece of information is a diameter, a height, or a width of the machine-readable second reference marker, alternatively wherein the first geometric piece of information is a length and/or an orientation of a certain marker surface boundary of the machine-readable first reference marker and the second geometric piece of information is a length and/or an orientation of a corresponding marker surface boundary of the machine-readable second reference marker, alternatively wherein the first geometric piece of information is a distance or a vector between the first reference point and the third reference point and the second geometric piece of information is a distance or a vector between the second reference point and the fourth reference point.
